(19) United States
(12) Patent Application Publication
(10) Pub. No.: US 2010/0158962 A1
(43) Pub. Date: Jun. 24, 2010

(54) CHARGING/SEPARATING COSMETIC MAKEUP COMPOSITIONS FOR KERATIN FIBERS

(75) Inventors: Karl Pays, Sanit-Maurice (FR); Yohann Bichon, Paris (FR); Sandrine Olivier, L'hay-Les-Roses (FR)

Correspondence Address: FINNEGAN, HENDERSON, FARABOW, GARRETT & DUNNER;LLP, 901 NEW YORK AVENUE, NW, WASHINGTON, DC, 20001-4413, US

(73) Assignee: L'Oreal S.A.

(21) Appl. No.: 12/635,621

(22) Filed: Dec. 10, 2009

Related U.S. Application Data
(63) Continuation of application No. 10/650,836, filed on Aug. 29, 2003, now abandoned.
(60) Provisional application No. 60/413,865, filed on Sep. 27, 2002.
(60) Provisional application No. 60/413,743, filed on Sep. 27, 2002.

(30) Foreign Application Priority Data
Sep. 6, 2002 (FR) 02/11102
Sep. 6, 2002 (FR) 02/11103

(51) Int. Cl. A61K 8/81; A61K 8/02; A61Q 1/00

(52) U.S. Cl. 424/401; 424/70.16

(57) ABSTRACT

The present invention relates to a cosmetic composition for making up keratin fibers, comprising a waxy phase containing at least one aqueous dispersion of wax particles, having a plateau Gp modulus of rigidity of greater than or equal to 4,000 Pa and the wax particles of said waxy phase having a size, expressed as the mean “effective” diameter by volume D[4.3], of less than or equal to 1 μm.

### CROSS-REFERENCE TO PRIORITY/PROVISIONAL APPLICATIONS

This is a continuation of application Ser. No. 10/650,836, filed Aug. 29, 2003, which claims priority under 35 U.S.C. §119 of FR-02/11102, filed Sep. 6, 2002, and FR-02/11103, filed Sep. 6, 2002, and claims the benefit under 35 U.S.C. §119(e) of provisional applications Ser. No. 60/413,865, filed Sep. 27, 2002, and Ser. No. 60/413,743, filed Sep. 27, 2002, each hereby expressly incorporated by reference.

### CROSS-REFERENCE TO COMPANION APPLICATIONS

Our copending applications Ser. No. ______ [Attorney Docket No. 032487-006], Ser. No. ______ [Attorney Docket No. 032487-007] and Ser. No. ______ [Attorney Docket No. 032487-008], each filed concurrently herewith and each assigned to the assignee hereof.

### BACKGROUND OF THE INVENTION

1. Technical Field of the Invention

The present invention relates to making up keratin fibers, for instance the eyelashes, the eyebrows and the hair, and more particularly to making up the eyelashes.

2. Description of Background/Related/Prior Art

Eye makeup compositions, also known as mascaras for the eyelashes or eyeliners for the eyelids, generally consist of a wax or a mixture of waxes dispersed using at least one surfactant in an aqueous phase also containing water-soluble polymers and pigments.

It is generally by means of the qualitative and quantitative choice of the waxes and polymers that the desired application specificities for makeup compositions are adjusted, for instance their fluidity, their covering power and/or their curling power. Thus, it is possible to produce various compositions, which, when applied especially to the eyelashes, induce a variety of effects such as lengthening, curling and/or thickening (charging effect).

The present invention is more particularly directed towards proposing a composition that is useful for producing a heavy makeup result on keratin fibers and especially the eyelashes, which is also known as charging makeup. For the purposes of the present invention, the term “keratin fibers” covers the hair, the eyelashes and the eyebrows and also extends to synthetic wigs and false eyelashes.

With the makeup compositions that are currently available, this effect is generally obtained by superimposing several coats of the makeup composition onto the keratin fibers and more particularly the eyelashes. Moreover, in the particular case of the eyelashes, this effect is very often associated with an aggregation of several eyelashes together, i.e., a non-individualization of the eyelashes.

For obvious reasons, it would be advantageous to obtain this thickening effect in a single application while at the same time obtaining good separation of the eyelashes.

To do this, it would be particularly advantageous to have available a makeup composition that is sufficiently concentrated in dry matter to significantly charge the eyelashes from the very first time they come into contact with the said composition, and that also allows each eyelash to be coated separately.

Standard eye makeup compositions conventionally have a solids content of between 10% and 40% by weight. If it is desired to increase this solids content beyond this value, a problem of lack of fluidity is rapidly encountered. The makeup composition becomes too thick on application and no longer has the deformability required for it to be applied uniformly over the entire surface of the eyelashes.

### SUMMARY OF THE INVENTION

It has now unexpectedly been determined that it is possible to significantly increase the solids content of a makeup composition for keratin fibers and more particularly its wax content, while at the same time retaining satisfactory rheological properties, especially in terms of deformability and consistency at rest, with the proviso of the choice of a specific emulsifying system and of a specific wax particle size.

Advantageously, although the claimed compositions have an increased amount of dry matter compared with conventional compositions, they maintain a plateau Gp modulus of rigidity that is suitable for the strain required for them to be uniformly applied with a brush or a comb onto the surface of keratin fibers, and especially the eyelashes.

According to one of its aspects, one embodiment of the invention is thus a cosmetic composition for making up keratin fibers, comprising a waxy phase containing at least one aqueous dispersion of wax particles, characterized in that it has a plateau Gp modulus of rigidity of greater than or equal to 4,000 Pa and less than or equal to 60,000 Pa and in that the wax particles of the said waxy phase have a size, expressed as the mean “effective” diameter by volume D[4.3], of less than or equal to 1 μm.

The present invention is also directed towards a process for making up keratin fibers, characterized in that a composition in accordance with the invention is applied to the said fibers.

The invention also relates to the use of a composition in accordance with the invention to obtain a charging and, where appropriate, separating makeup result on keratin fibers, and especially the eyelashes and the eyebrows.

For the purposes of the present invention, the term “charging” is intended to qualify the notion of heavy makeup of the eyelashes.

### DETAILED DESCRIPTION OF BEST MODE AND SPECIFIC/PREFERRED EMBODIMENTS OF THE INVENTION

It has thus been found, unexpectedly, that it is possible to prepare makeup compositions with a high modulus of rigidity, i.e., greater than or equal to 4,000 Pa, in particular greater than or equal to 5,500 Pa and especially greater than or equal to 7,000 Pa, by using a specific emulsifying system and the formulation of the waxy phase in the form of a dispersion of particles of determined size in these compositions.

In particular, the composition according to the invention may have a plateau modulus of rigidity of less than 60,000 Pa, so as to give it a deformability that is sufficient for its application to the surface to be made up.

### Characterization of the Solids Content:

The solids content, i.e., the content of non-volatile matter, may be measured in various ways; examples that may be mentioned include oven-drying methods, drying methods by exposure to infrared radiation and also chemical methods by titration of the water according to Karl Fischer.

The solids content, commonly referred to as the “dry extract” of the compositions according to the invention, is preferably measured by heating the sample with infrared rays with a wavelength of 2 μm to 3.5 μm. The substances contained in the said compositions that have a high vapor pressure evaporate under the effect of this radiation. Measurement of the weight loss of the sample makes it possible to determine the “dry extract” of the composition. These measurements are performed using an LP16 commercial infrared desiccator from Mettler. This technique is fully described in the machine documentation supplied by Mettler.

The measuring protocol is as follows:

About 1 g of the composition is spread onto a metal crucible. After introducing this crucible into the desiccator, it is subjected to a set temperature of 120° C. for one hour. The wet mass of the sample, corresponding to the initial mass, and the dry mass of the sample, corresponding to the mass after exposure to the radiation, are measured using a precision balance.

The solids content is calculated in the following manner:

Dry extract=100×(dry mass/wet mass).

The compositions according to the invention are characterized by a solids content of greater than or equal to 48% by weight.

The compositions according to the invention are also especially characterized by a solids content of less than or equal to 42% by weight, especially greater than or equal to 45.5% by weight, or even greater than 48% by weight, relative to the total weight of the composition.

### Rheological Characteristics:

The compositions in accordance with the invention are characterized by viscoelastic behavior.

In general, a material is said to be viscoelastic when, under the effect of shear, it has both the characteristics of a purely elastic material, i.e., capable of storing energy, and the characteristics of a purely viscous material, i.e., capable of dissipating energy.

More particularly, the viscoelastic behavior of the compositions in accordance with the invention may be characterized by its modulus of rigidity G, its elasticity δ and its flow threshold τ_{c} these parameters are defined especially in the publication “Initiation a la rhéologie [Introduction to Rheology]”, G. Couarraze and J. L. Grossiord, 2nd edition, 1991, published by Lavoisier-Tec 1 Doc.

These parameters are determined by means of measurements performed at 25° C.±0.5° C. using a Haake RheoStress 600® controlled-stress rheometer from the company ThermoRhéo, equipped with a stainless-steel rotor with plate/plate geometry, the plate having a diameter of 20 mm and a gap (distance between the lower plate—known as the stator plate—on which the composition is deposited, and the upper plate—known as the rotor plate) of 0.3 mm. The two plates are striated to limit the sliding phenomena to the walls of the plates.

The dynamic measurements are performed by applying a hannonic variation of the stress. In these experiments, the magnitudes of the shear, the shear rate and the stress are low so as to remain within the limits of the linear viscoelastic domain of the material (conditions for evaluating the rheological characteristics of the composition at rest).

The linear viscoelastic domain is generally defined by the fact that the response of the material (i.e., the strain) is at any moment directly proportional to the value of the applied force (i.e., the stress). In this domain, the applied stresses are small and the material undergoes strains without modifying its microscopic structure. Under these conditions, the material is studied “at rest” and non-destructively.

The composition is subjected to a harmonic shear according to a stress τ(t) varying sinusoidally according to a pulse ω(ω=2IIν, ν being the frequency of the applied shear). The composition thus sheared undergoes a stress τ(t) and responds according to a strain γ(t) corresponding to micro-strains for which the modulus of rigidity varies little as a function of the imposed stress.

The stress τ(t) and the strain γ(t) are defined, respectively, by the following relationships:

τ(t)=τ₀ cos(ω·t) γ(t)=γ₀ cos(ω·t−δ)

τ₀ being the maximum amplitude of the stress and γ0 being the maximum amplitude of the strain. δ is the dephasing angle between the stress and the strain.

The measurements are performed at a frequency of 1 Hz (ν=1 Hz).

The change in the modulus of rigidity G (corresponding to the ratio of τ₀ to γ₀) and in the elasticity δ (corresponding to the dephasing angle of the applied stress relative to the measured strain) as a function of the applied stress τ(t) are thus measured.

The strain of the composition is measured in particular for the stress region in which the variation of the modulus of rigidity G and of the elasticity δ is less than 7% (micro-strain zone), and the “plateau” parameters Gp and δₚ, are thus determined. The threshold stress τ_{c} (corresponding to the minimum force that it is necessary to apply to the composition to cause it to flow) is determined from the curve δ=f(τ) and corresponds to the value of τ for which δ(τ_{c})=1.05δₚ.

The viscoelastic behavior of the compositions according to the invention is especially characterized by a plateau Gp modulus of rigidity of greater than or equal to 4,000 Pa, which may especially be greater than or equal to 5,500 Pa, and which may in particular be greater than or equal to 7,000 Pa or even greater than or equal to 10,000 Pa.

In particular, the compositions according to the invention have a plateau Gp modulus of rigidity of less than 60,000 Pa or even less than 50,000 Pa, especially less than 40,000 Pa and in particular less than 30,000 Pa.

In addition, the compositions according to the invention may have a plateau elasticity δₚ that may range from 1° to 45° and better still ranging from 5° to 30°.

The compositions in accordance with the invention may moreover have a flow threshold τ_{c} ranging from 5 Pa to 3,500 Pa and preferably ranging from 20 Pa to 1,000 Pa, which means that the composition according to the invention does not flow under its own weight, but rather that it is necessary to apply a minimum stress to the composition to make it flow.

### Wax:

The compositions according to the invention comprise a waxy phase containing at least one aqueous dispersion of particles of a wax or of a mixture of waxes.

For the purposes of the present invention, the wax or the mixture of waxes present in the composition according to the invention will be referred to by the general term “waxy phase”.

The wax under consideration in the context of the present invention is generally a lipophilic compound that is solid at room temperature (25 ° C.), with a solid/liquid reversible change of state, having a melting point of greater than or equal to 30° C., which may be up to 120° C.

By bringing the wax to the liquid form (melting), it is possible to make it miscible with oils and to form a microscopically uniform mixture, but on cooling the mixture to room temperature, recrystallization of the wax in the oils of the mixture is obtained.

In particular, the waxes that are suitable for the invention may have a melting point of greater than about 45° C. and in particular greater than 55° C.

The melting point of the wax may be measured using a differential scanning calorimeter (D.S.C.), for example the calorimeter sold under the name DSC 30 by Mettler.

The measuring protocol is as follows:

A sample of 15 mg of product placed in a crucible is subjected to a first temperature rise ranging from 0° C. to 120° C., at a heating rate of 10° C./minute, it is then cooled from 120° C. to 0° C. at a cooling rate of 10° C./minute and is finally subjected to a second temperature increase ranging from 0° C. to 120° C. at a heating rate of 5° C./minute. During the second temperature increase, the variation of the difference in power absorbed by the empty crucible and by the crucible containing the sample of product is measured as a function of the temperature. The melting point of the compound is the temperature value corresponding to the top of the peak of the curve representing the variation in the difference in absorbed power as a function of the temperature.

The waxes that may be used in the compositions according to the invention are chosen from waxes that are solid and rigid at room temperature, of animal, plant, mineral or synthetic origin, and mixtures thereof.

The wax may also have a hardness ranging from 0.05 MPa to 15 MPa and preferably ranging from 6 MPa to 15 MPa. The hardness is determined by measuring the compressive strength, measured at 20° C. using a texturometer sold under the name TA-XT2i by Rheo, equipped with a stainless-steel cylinder 2 mm in diameter traveling at a measuring speed of 0.1 mm/s, and penetrating into the wax to a penetration depth of 0.3 mm.

The measuring protocol is as follows:

The wax is melted at a temperature equal to the melting point of the wax+20° C. The molten wax is cast in a container 30 mm in diameter and 20 mm deep. The wax is recrystallized at room temperature (25° C.) over 24 hours and is then stored for at least 1 hour at 20° C. before performing the hardness measurement. The value of the hardness is the compressive strength measured divided by the area of the texturometer cylinder in contact with the wax.

Hydrocarbon-based waxes such as beeswax, lanolin wax and Chinese insect waxes; rice wax, carnauba wax, candelilla wax, ouricury wax, esparto grass wax, cork fiber wax, sugar cane wax, Japan wax and sumach wax; montan wax, microcrystalline waxes, paraffins and ozokerite; polyethylene waxes, the waxes obtained by Fisher-Tropsch synthesis and waxy copolymers, and also esters thereof, may especially be used.

The waxes obtained by catalytic hydrogenation of animal or plant oils containing linear or branched C₈-C₃₂ fatty chains, may also be mentioned.

Among these oils, mention may be made especially of hydrogenated jojoba oil, isomerized jojoba oil such as the trans-isomerized partially hydrogenated jojoba oil manufactured or sold by Desert Whale under the trademark Iso-Jojoba-50®, hydrogenated sunflower oil, hydrogenated castor oil, hydrogenated coconut oil and hydrogenated lanolin oil, bis(1,1,1-trimethylolpropane)tetrastearate sold under the name “Hest 2T-4S” by Heterene, and bis(1,1,1-trimethylolpropane)tetrabehenate sold under the name Hest 2T-4B by Heterene.

Silicone waxes and fluoro waxes may also be mentioned.

The wax obtained by hydrogenation of olive oil esterified with stearyl alcohol, sold under the name “Phytowax Olive 18 L 57”, or the waxes obtained by hydrogenation of castor oil esterified with cetyl alcohol, sold under the name “Phytowax Ricin 16L64 and 22L73” by Sophim, may also be used. Such waxes are described in FR-A-2,792,190.

The composition according to the invention generally contains from 0.1% to 40% by weight of wax(es); it may in particular contain from 5% to 40%, more particularly from 20% to 40% and better still from 30% to 40% by weight relative to the total weight of the composition.

The wax or mixture of waxes or even the aqueous phase is present in the compositions according to the invention in the form of an aqueous dispersion of particles whose size, expressed as the mean “effective” diameter by volume D[4.3] as defined below, is less than or equal to 1 μm.

The wax particles may have various shapes. They may especially be spherical.

### Characterization of the Particle Sizes:

The particle sizes may be measured by various techniques; mention may be made in particular of light-scattering techniques (dynamic and static), Coulter counter methods, sedimentation rate measurements (related to the size via Stokes' law) and microscopy. These techniques make it possible to measure a particle diameter and, for some of them, a particle size distribution.

The sizes and size distributions of the particles in the compositions according to the invention are preferably measured by static light scattering using a commercial granulometer such as the MasterSizer 2000 from Malvern. The data are processed on the basis of the Mie scattering theory. This theory, which is exact for isotropic particles, makes it possible to determine an “effective” particle diameter in the case of non-spherical particles. This theory is described especially in the publication by Van de Hulst, H. C., “Light Scattering by Small Particles,” Chapters 9 and 10, Wiley, New York, 1957.

The composition is characterized by its mean “effective” diameter by volume D[4.3], defined in the following manner:

$$D [ 4.3 ] = \frac{{\sum }_{i} {V}_{i} \cdot {d}_{i}}{{\sum }_{i} {V}_{i}}$$

in which Vᵢ represents the volume of the particles with an effective diameter dᵢ. This parameter is described especially in the technical documentation of the granulometer.

The measurements are performed at 25° C. on a dilute particle dispersion, obtained from the composition in the following manner: 1) dilution by a factor of 100 with water, 2) homogenization of the solution, 3) standing of the solution for 18 hours, 4) recovery of the whitish uniform supernatant.

The “effective” diameter is obtained by taking a refractive index of 1.33 for water and a mean refractive index of 1.42 for the particles.

The wax particles of the waxy phase in the compositions according to the invention may be characterized by a size, expressed as a mean “effective” diameter by volume D[4.3], of less than or equal to 1 μm, especially less than or equal to 0.75 μm and better still less than or equal to 0.55 μm.

The particle size is mainly linked to the nature of the emulsifying system used to prepare the dispersion.

### Emulsifying System:

According to the invention, an emulsifier appropriately chosen to obtain an oil-in-water emulsion is generally used. In particular, an emulsifier having at 25° C. an HLB (hydrophilic-lipophilic balance), in the Griffin sense, of greater than or equal to 8 may be used.

The HLB value according to Griffin is defined in J. Soc. Cosm. Chem. 1954 (volume 5), pages 249-256.

The compositions according to the invention may especially contain emulsifying surfactants present especially in a proportion ranging from 0.1% to 40% by weight and better still from 0.5% to 20% by weight relative to the total weight of the composition.

These surfactants may be chosen from nonionic, anionic, cationic and amphoteric surfactants or emulsifying surfactants. Reference may be made to the document “Encyclopedia of Chemical Technology, Kirk-Othmer”, volume 22, p. 333-432, 3rd edition, 1979, Wiley, for the definition of the properties and (emulsifying) functions of surfactants, in particular pp. 347-3 77 of this reference, for anionic, amphoteric and nonionic surfactants.

In the context of the present invention, this emulsifying system may comprise at least one ionic surfactant and/or one nonionic surfactant with an HLB of greater than or equal to 8 at 25° C. or at least one of these two surfactants combined with at least one gelling polymer.

Nonionic Surfactant with an HLB of Greater than or Equal to 8:

As non-limiting illustrations of nonionic surfactants with an HLB of greater than or equal to 8 which may be used, alone or as a mixture, in the makeup compositions according to the invention, mention may be made especially of:

oxyethylenated and/or oxypropylenated ethers (which may comprise from 1 to 150 oxyethylene and/or oxypropylene groups) of glycerol;

oxyethylenated and/or oxypropylenated ethers (which may comprise from 1 to 150 oxyethylene and/or oxypropylene groups) of fatty alcohols (especially of C₈-C₂₄ and preferably C₁₂C₁₈ alcohol), such as oxyethylenated cetearyl alcohol ether containing 30 oxyetbylene groups (CTFA name “Ceteareth-30”) and the oxyethylenated ether of the mixture of C₁₂-C₁₅ fatty alcohols comprising 7 oxyethylene groups (CTFA name “C 12-15 Pareth-7” sold under the name “Neodol 25-7®” by Shell Chemicals),

fatty acid esters (especially of a C₈-C₂₄ and preferably C₁₆-C₂₂ acid) of polyethylene glycol (which may comprise from 1 to 150 ethylene glycol units), such as PEG-50 stearate and PEG-40 monostearate sold under the name Myrj 52P by ICI Uniqema,

fatty acid esters (especially of a C₈-C₂₄ and preferably C₁₆-C₂₂ acid) of oxyethylenated and/or oxypropylenated glyceryl ethers (which may comprise from 1 to 150 oxyethylene and/or oxypropylene groups), for instance PEG-200 glyceryl monostearate sold under the name “Simulsol 220™” by SEPPIC; glyceryl stearate polyethoxylated with 30 ethylene oxide groups, for instance the product Tagat S sold by Goldschmidt, glyceryl oleate polyethoxylated with 30 ethylene oxide groups, for instance the product Tagat ◯ sold by Goldschmidt, glyceryl cocoate polyethoxylated with 30 ethylene oxide groups, for instance the product Varionic LI 13 sold by Sherex, glyceryl isostearate polyethoxylated with 30 ethylene oxide groups, for instance the product Tagat L sold by Goldschmidt, and glyceryl laurate polyethoxylated with 30 ethylene oxide groups, for instance the product Tagat I from the company Goldschmidt,

fatty acid esters (especially of a C₈-C₂₄ and preferably C₁₆-C₂₂ acid) of oxyethylenated and/or oxypropylenated sorbitol ethers (which may comprise from 1 to 150 oxyethylene and/or oxypropylene groups), for instance polysorbate 60 sold under the name “Tween 60” by Uniqema,

dimethicone copolyol, such as the product sold under the name “Q2-5220” by Dow Corning,

dimethicone copolyol benzoate (Finsolv SLB 101 and 201 by Finetex),

copolymers of propylene oxide and of ethylene oxide, also known as EO/PO polycondensates,

and mixtures thereof

The EO/PO polycondensates are more particularly copolymers consisting of polyethylene glycol and polypropylene glycol blocks, for instance polyethylene glycol/polypropylene glycol/polyethylene glycol triblock polycondensates. These triblock polycondensates have, for example, the following chemical structure:

H—(O—CH₂—CH₂)ₐ—(O—CH(CH₃)—CH₂)_{b}—(O—CH₂—CH₂)ₐ—OH,

in which formula a ranges from 2 to 120 and b ranges from 1 to 100.

The EO/PO polycondensate preferably has a weight-average molecular weight ranging from 1,000 to 15,000 and better still ranging from 2,000 to 13,000.

Advantageously, the said EO/PO polycondensate has a cloud point, at 10 g/l in distilled water, of greater than or equal to 20° C. and preferably greater than or equal to 60° C. The cloud point is measured according to ISO standard 1065. As EO/PO polycondensates that may be used according to the invention, mention may be made of the polyethylene glycol/polypropylene glycol/polyethylene glycol triblock polycondensates sold under the name “Synperonic”, for instance “Synperonic PE/L44” and “Synperonic PE/F 127”, by ICI.

One or more nonionic surfactants with an HLB of less than 8 at 25° C. may, where appropriate, be combined with this nonionic surfactant with an HLB of greater than or equal to 8.

As non-limiting illustrations of these agents with an HLB of less than 8 at 25° C., mention may be made more particularly of:

saccharide esters and ethers, such as sucrose stearate, sucrose cocoate and sorbitan stearate, and mixtures thereof, for instance Arlatone 2121 sold by ICI;

fatty acid esters (especially of a C₈-C₂₄ and preferably C₁₆-C₂₂ acid) of polyols, especially of glycerol or of sorbitol, such as glyceryl stearate, glyceryl stearate such as the product sold under the name Tegin M by Goldschmidt, glyceryl laurate such as the product sold under the name Imwitor 312 by Hüls, polyglyceryl-2 stearate, sorbitan tristearate or glyceryl ricinoleate;

the mixture of cyclomethicone/dimethicone copolyol sold under the name “Q2-3225C” by Dow Corning.

The amount of nonionic surfactant is generally adjusted so as to obtain a composition having the parameters as defined above. The determination of this amount falls within the competence of a person skilled in the art.

As non-limiting illustration of the scope of the invention, this amount of nonionic surfactant with an HLB of greater than or equal to 8 may range from 0.01% to 40% by weight, in particular from 0.1% to 20% or even from 0.5% to 15% and better still from 0.5% to 10% by weight, relative to the total weight of the composition.

### Ionic Surfactant:

In general, the composition claimed contains an ionic surfactant especially in combination with at least one nonionic surfactant with an HLB of greater than or equal to 8 and/or at least one gelling polymer.

The ionic surfactants used in the context of the present invention may be anionic or cationic. However, the choice of at least one anionic surfactant is generally favored.

As illustrations of anionic surfactants that are suitable for the invention, mention may be made more particularly of:

C₁₆-C₃₀ fatty acid salts, especially those derived from amines, for instance triethanolamine stearate;

polyoxyethylenated fatty acid salts, especially those derived from amines or alkali metal salts, and mixtures thereof;

phosphoric esters and salts thereof, such as “DEA oleth-10 phosphate” (Crodafos N 10N from the company Croda);

sulphosuccinates such as “Disodium PEG-S citrate lauryl sulphosuccinate” and “Disodium ricinoleamido MEA sulphosuccinate”;

alkyl ether sulphates, such as sodium lauryl ether sulphate;

isethionates;

acylglutamates such as “Disodium hydrogenated tallow glutamate” (Amisoft HS-21 R sold by Ajinomoto), and mixtures thereof.

Triethanolamine stearate is most particularly suitable for the invention. This surfactant is generally obtained by simple mixing of stearic acid and triethanolamine.

Illustrations of cationic surfactants that may especially be mentioned include:

alkylimidazolidiniums, such as isostearylethylirnidonium ethosulphate,

ammonium salts, such as N,N,N-trimethyl-1-docosanaminium chloride (behentrimonium chloride).

The compositions according to the invention may also contain one or more amphoteric surfactants, for instance N-acylamino acids such as N-alkylaminoacetates and disodium cocoamphodiacetate, and amine oxides such as stearamine oxide, or alternatively silicone surfactants, for instance dimethicone copolyol phosphates such as the product sold under the name “Pecosil PS 100” by Phoenix Chemical.

In general, the compositions according to the invention may contain from 0.01% to 30% by weight, in particular from 0.1% to 15% by weight or even from 0.5% to 10% by weight of ionic surfactant, relative to the total weight of the composition.

### Gelling Polymer:

The compositions according to the invention may also contain at least one gelling polymer.

According to the present invention, the term “gelling polymer” means a polymer that is capable of gelling the continuous phase, generally the aqueous phase, of the compositions according to the invention.

The gelling polymer that may be used according to the invention may be characterized especially by its capacity to form in water, above a certain concentration C*, a gel characterized by oscillatory rheology (ν=1 Hz) by a flow threshold τ_{c} at least equal to 10 Pa. This concentration may vary within a wide range depending on the nature of the gelling agent under consideration.

By way of illustration, this concentration is between 1% and 2% by weight for an acrylamide/sodium 2-acrylamidomethylpropanesulphonate copolymer as an inverse emulsion at 40% in polysorbate 80/I-C16, for instance the product sold under the name “Simulgel 600” by SEPPIC, and is about 0.5% by weight for an AMPS/ethoxylated (25 EO) cetearyl methacrylate copolymer crosslinked with trimethylolpropane triacrylate (TMPTA).

The gelling polymer may be a water-soluble polymer and is thus present in the aqueous phase of the composition in dissolved form.

This gelling polymer may be chosen more particularly from:

homopolymers or copolymers of acrylic or methacrylic acid or the salts and esters thereof, and in particular the products sold under the names “Versicol F” or “Versicol K” by Allied Colloid, “Ultrahold 8” by Ciba-Geigy, and the polyacrylic acids of Synthalen K type;

copolymers of acrylic acid and of acrylamide sold in the form of the sodium salt thereof under the names “Reten” by Hercules, sodium polymethacrylate sold under the name “Darvan 7” by Vanderbilt, and the sodium salts of polyhydroxycarboxylic acids sold under the name “Hydagen F” by Henkel;

polyacrylic acid/alkyl acrylate copolymers of the Pemulen type;

AMPS (polyacrylamidomethylpropanesulphonic acid partially neutralized with ammonia and highly crosslinked) sold by Clariant;

AMPS/acrylamide copolymers of the Sepigel or Simulgel type, sold by SEPPIC, and

AMPS/polyoxyethylenated alkyl methacrylate copolymers (crosslinked or non-crosslinked), and mixtures thereof.

As other examples of water-soluble gelling polymers, mention may be made of:

proteins, for instance proteins of plant origin such as wheat or soybean proteins; proteins of animal origin such as keratins, for example keratin hydrolysates and sulphonic keratins;

anionic, cationic, amphoteric or nonionic chitin or chitosan polymers;

cellulose polymers such as hydroxyethylcellulose, hydroxypropylcellulose, methylcellulose, ethylhydroxyethylcellulose and carboxymethylcellulose, and also quatemized cellulose derivatives;

vinyl polymers, for instance polyvinylpyrrolidones, copolymers of methyl vinyl ether and of maleic anhydride, the copolymer of vinyl acetate and of crotonic acid, copolymers of vinylpyrrolidone and of vinyl acetate; copolymers of vinylpyrrolidone and of caprolactam; polyvinyl alcohol;

polymers of natural origin, optionally modified, such as:

gum arabics, guar gum, xanthan derivatives and karaya gum

alginates and carrageenans;

glycosaminoglycans, and hyaluronic acid and its derivatives;

shellac resin, sandarac gum, dammar resins, elemi gums and copal resins;

deoxyribonucleic acid;

mucopolysaccharides such as hyaluronic acid and chondroitin sulphate, and mixtures thereof.

The gelling polymer is generally present in the composition in an amount that is sufficient to adjust the modulus of rigidity to a value of greater than or equal to 4,000 Pa, in particular greater than or equal to 5,500 Pa, or even greater or equal to 7,000 Pa.

In this instance, the gelling polymer may be present in the composition according to the invention in a solids content ranging from 0.1% to 60% by weight, preferably from 0.5% to 40% by weight and better still from 1% to 30% by weight, or even from 5% to 20% by weight, relative to the total weight of the composition.

It is understood that this amount is moreover liable to vary depending on whether or not the said polymer is combined with an ionic and/or nonionic surfactant and/or a film-forming agent, which are themselves also capable of acting on the consistency of the said composition.

### Film-Forming Polymer:

The composition according to the invention may also comprise a film-forming agent.

According to the present invention, the term “film-forming polymer” means a polymer that is capable, by itself or in the presence of an auxiliary film-forming agent, of forming a continuous film that adheres to a support, especially to keratin materials.

Among the film-forming polymers that may be used in the composition of the present invention, mention may be made of synthetic polymers, of free-radical type or of polycondensate type, and polymers of natural origin, and mixtures thereof.

The film-forming polymers of free-radical type may especially be vinyl polymers or copolymers, especially acrylic polymers.

The vinyl film-forming polymers may result from the polymerization of ethylenically unsaturated monomers containing at least one acid group and/or esters of these acid monomers and/or amides of these acid monomers, for instance α,β-ethylenic unsaturated carboxylic acids such as acrylic acid, methacrylic acid, crotonic acid, maleic acid or itaconic acid.

The vinyl film-forming polymers may also result from the homopolymerization or copolymerization of monomers chosen from vinyl esters, for instance vinyl acetate, vinyl neodecanoate, vinyl pivalate, vinyl benzoate and vinyl t-butylbenzoate, and styrene monomers, for instance styrene and α-methyistyrene.

Among the film-forming polycondensates that may be mentioned are polyurethanes, polyesters, polyesteramides, polyamides and polyureas.

The optionally modified polymers of natural origin may be chosen from shellac resin, sandarac gum, dammar resins, elemi gums, copal resins and cellulose-based polymers, and mixtures thereof.

The film-forming polymer may be present in the form of particles in aqueous dispersion, which are generally known as latices or pseudolatices. The techniques for preparing these dispersions are well known to those skilled in the art.

Aqueous dispersions of film-forming polymers that may be used include the acrylic dispersions sold under the names Neocryl XK-90®, Neocryl A-1070®, Neocryl A-1090®, Neocryl BT-62®, Neocryl A-1079® and Neocryl A-523® by Avecia-Neoresins, Dow Latex 432® by Dow Chemical, Daitosol 5000 AD®, by Daito Kasey Kogyo; or the aqueous dispersions of polyurethane sold under the names Neorez R981® and Neorez R-974® by Avecia-Neoresins, Avalure UR-405®, Avalure UR-410®, Avalure UR-425®, Avalure UR-450®, Sancure 875®, Sancure 861®, Sancure 878® and Sancure 2060® by Goodrich, Impranil 85® by Bayer, Aquamere H-1511® by Hydromer; the sulphopolyesters sold under the brand name Eastman AQ by Eastman Chemical Products.

The composition according to the invention may also comprise an auxiliary film-forming agent that promotes the formation of a film with the film-forming polymer.

### Physiologically Acceptable Medium:

Generally, the compositions according to the invention are based on water or on an aqueous medium, i.e., a mixture of water with at least one organic solvent.

The aqueous medium of the composition may thus comprise a mixture of water and of water-miscible organic solvent, for instance lower monoalcohols containing from 1 to 5 carbon atoms such as ethanol and isopropanol, glycols containing from 2 to 8 carbon atoms, such as glycerol, propylene glycol, ethylene glycol, 1,3-butylene glycol and dipropylene glycol, C₃-C₄ ketones and C₂-C₄ aldehydes. The aqueous medium (water and the optional water-miscible organic solvent) may represent, in practice, from 5% to 90% by weight relative to the total weight of the composition.

### Additives:

The compositions claimed may also contain ingredients commonly used in the field of makeup for keratin fibers.

The composition according to the invention may especially comprise one or more oils.

The oil may be chosen from volatile oils and/or non-volatile oils, and mixtures thereof. The composition advantageously comprises at least one volatile oil.

For the purposes of the invention, the term “volatile oil” means an oil that is capable of evaporating on contact with the skin or the keratin fiber in less than one hour, at room temperature and atmospheric pressure.

The volatile organic solvent(s) and volatile oils of the invention are volatile organic solvents and cosmetic oils that are liquid at room temperature, with a non-zero vapour pressure at room temperature and atmospheric pressure, ranging in particular from 0.13 Pa to 40,000 Pa (10⁻³ to 300 mmHg), in particular ranging from 1.3 Pa to 13,000 Pa (0.01 to 100 mmHg), and more particularly ranging from 1.3 Pa to 1,300 Pa (0.01 to 10 mmHg).

The term “non-volatile oil” means an oil that remains on the skin or the keratin fiber at room temperature and atmospheric pressure for at least several hours and that especially has a vapour pressure of less than 10⁻³ mmHg (0.13 Pa).

These oils may be hydrocarbon-based oils, silicone oils or fluoro oils, or mixtures thereof.

The term “hydrocarbon-based oil” means an oil mainly containing hydrogen and carbon atoms and optionally oxygen, nitrogen, sulphur or phosphorus atoms. The volatile hydrocarbon-based oils may be chosen from hydrocarbon-based oils containing from 8 to 16 carbon atoms, and especially branched C₈-C₁₆ alkanes, for instance C₈-C₁₆ isoalkanes of petroleum origin (also known as isoparaffins), for instance isododecane (also known as 2,2,4,4,6-pentamethylheptane), isodecane and isohexadecane, for example the oils sold under the trademarks Isopar or Permetyl, branched C₈-C₁₆ esters and isohexyl neopentanoate, and mixtures thereof. Other volatile hydrocarbon-based oils, for instance petroleum distillates, especially those sold under the name Shell Solt by Shell, may also be used. The volatile solvent is preferably chosen from volatile hydrocarbon-based oils containing from 8 to 16 carbon atoms, and mixtures thereof.

Volatile oils that may also be used include volatile silicones, for instance volatile linear or cyclic silicone oils, especially those with a viscosity≦8 centistokes (8×10⁻⁶ m²/s) and especially containing from 2 to 7 silicon atoms, these silicones optionally comprising alkyl or alkoxy groups containing from 1 to 10 carbon atoms. As volatile silicone oils that may be used in the invention, mention may be made especially of octamethyl cyclotetrasiloxane, decamethyl cyclopentasiloxane, dodecamethyl cyclohexasiloxane, heptamethyl hexyltrisiloxane, heptamethyloctyl trisiloxane, hexamethyl disiloxane, octamethyl trisiloxane, decamethyl tetrasiloxane and dodecamethyl pentasiloxane, and mixtures thereof.

Volatile fluorinated solvents such as nonafluoromethoxybutane or perfluoromethylcyclopentane may also be used.

The volatile oil may be present in the composition according to the invention in a content ranging from 0.1% to 60% by weight and preferably from 0.1% to 30% by weight relative to the total weight of the composition.

The composition may also comprise at least one non-volatile oil chosen especially from non-volatile hydrocarbon-based oils and/or silicone oils and/or fluoro oils.

Non-volatile hydrocarbon-based oils that may especially be mentioned include:

hydrocarbon-based oils of plant origin, such as triglycerides consisting of fatty acid esters of glycerol, the fatty acids of which may have varied chain lengths from C₄ to C₂₄, these chains possibly being linear or branched, and saturated or unsaturated; these oils are especially wheatgerm oil, sunflower oil, grapeseed oil, sesame seed oil, maize oil, apricot oil, castor oil, karite oil, avocado oil, olive oil, soybean oil, sweet almond oil, palm oil, rapeseed oil, cottonseed oil, hazelnut oil, macadamia oil, jojoba oil, alfalfa oil, poppy oil, pumpkin oil, marrow oil, blackcurrant oil, evening primrose oil, millet oil, barley oil, quinoa oil, rye oil, safflower oil, candlenut oil, passion flower oil and musk rose oil; or alternatively caprylic/capric acid triglycerides such as those sold by Stearineries Dubois or those sold under the names Miglyol 810, 812 and 818 by Dynamit Nobel,

synthetic ethers containing from 10 to 40 carbon atoms;

linear or branched hydrocarbons of mineral or synthetic origin, such as petroleum jelly, polydecenes, hydrogenated polyisobutene such as parleam, and squalane, and mixtures thereof;

synthetic esters such as oils of formula R₁COOR₂ in which R₁ represents a linear or branched fatty acid residue containing from 1 to 40 carbon atoms and R₂ represents an in particular branched hydrocarbon-based chain containing from 1 to 40 carbon atoms, on condition that R₅+R₆≧10, such as, for example, purcellin oil (cetostearyl octanoate), isopropyl myristate, isopropyl palmitate, C₁₂-C₁₅ alkyl benzoate, hexyl laurate, diisopropyl adipate, isononyl isononanoate, 2-ethylhexyl palmitate, isostearyl isostearate, alkyl or polyalkyl octanoates, decanoates or ricinoleates such as propylene glycol dioctanoate; hydroxylated esters such as isostearyl lactate and diisostearyl malate; and pentaerythritol esters;

fatty alcohols that are liquid at room temperature, containing a branched and/or unsaturated carbon-based chain containing from 12 to 26 carbon atoms, for instance octyldodecanol, isostearyl alcohol, oleyl alcohol, 2-hexyldecanol, 2-butyloctanol or 2-undecylpentadecanol;

higher fatty acids such as oleic acid, linoleic acid or linolenic acid; and

mixtures thereof.

The non-volatile silicone oils that may be used in the composition according to the invention may be non-volatile polydimethylsiloxanes (PDMSs), polydimethylsiloxanes comprising alkyl or alkoxy groups, that are pendent and/or at the end of a silicone chain, the groups each containing from 2 to 24 carbon atoms, phenylsilicones, for instance phenyltrimethicones, phenyldimethicones, phenyltrimethylsiloxydiphenylsiloxanes, diphenyldimethicones, diphenylmethyldiphenyltrisiloxanes and 2-phenylethyltrimethyl-siloxysilicates.

The fluoro oils that may be used in the invention are, in particular, fluorosilicone oils, fluoropolyethers or fluorosilicones, as described in EP-A-847,752.

The non-volatile oils may be present in the composition according to the invention in a content ranging from 0.1% to 20% by weight and preferably from 0.1% to 12% by weight relative to the total weight of the composition.

The composition may also comprise other ingredients usually used in cosmetics. Such ingredients may especially be coalescers, fillers, dyestuffs, for instance pigments, nacres or liposoluble or water-soluble dyes, goniochromatic dyes, preserving agents, oils, moisturizers and fragrances, and mixtures thereof, which are well known in the prior art.

The pigments may be white or colored, and mineral and/or organic; they are insoluble in the physiologically acceptable medium of the composition. Among the mineral pigments that may be mentioned are titanium dioxide, optionally surface-treated, zirconium oxide or cerium oxide, and also iron oxide or chromium oxide, manganese violet, ultramarine blue, chromium hydrate and ferric blue, and mixtures thereof. Among the organic pigments that may be mentioned are carbon black, pigments of D & C type, and lakes based on cochineal carmine or on barium, strontium, calcium or aluminum, and mixtures thereof. Pigments with a particular optical effect, for instance glass particles coated with metal, especially with gold, silver and platinum, may also be used.

The nacres or nacreous pigments are iridescent particles produced especially by certain molluscs in their shell or else synthesized, which are insoluble in the physiologically acceptable medium of the composition. They may be chosen from white nacreous pigments such as mica coated with titanium or with bismuth oxychloride, colored nacreous pigments such as titanium mica with iron oxides, titanium mica with, especially, ferric blue or chromium oxide, titanium mica with an organic pigment of the abovementioned type, and also nacreous pigments based on bismuth oxychloride, and mixtures thereof. Interference pigments, especially liquid-crystal or multilayer interference pigments, may also be used.

The dyestuffs may represent from 0.01% to 30% by weight, preferably from 0.1% to 25% by weight and better still from 1% to 20% by weight, relative to the total weight of the composition.

The fillers may be chosen from those that are well known to a person skilled in the art and commonly used in cosmetic compositions. The fillers may be mineral or organic, and lamellar or spherical. Mention may be made of talc, mica, silica, kaolin, powders of polyamide, for instance Nylon® (Orgasol from Atochem), of poly-β-alanine and of polyethylene, powders of tetrafluoroethylene polymers, for instance Teflon®, lauroyllysine, starch, boron nitride, hollow polymer microspheres such as those of polyvinylidene chloride/acrylonitrile, for instance Expancel® (Nobel Industrie), acrylic powders such as Polytrap® (Dow Corning), polymethyl methacrylate particles and silicone resin microbeads (for example Tospearlse from Toshiba), precipitated calcium carbonate, magnesium carbonate and magnesium hydrocarbonate, hydroxyapatite, hollow silica microspheres (Silica Beads® from Maprecos), glass or ceramic microcapsules, metal soaps derived from organic carboxylic acids containing from 8 to 22 carbon atoms and preferably from 12 to 18 carbon atoms, for example zinc stearate, magnesium stearate, lithium stearate, zinc laurate or magnesium myristate.

The fillers may represent from 0.1% to 25% and better still from 1% to 20% by weight relative to the total weight of the composition.

The composition according to the invention may also comprise fibers to allow an improvement in the lengthening effect.

The term “fiber” should be understood as meaning an object of length L and diameter D such that L is very much greater than D, D being the diameter of the circle in which the cross section of the fiber is inscribed. In particular, the ratio L/D (or shape factor) is chosen in the range from 3.5 to 2,500, especially from 5 to 500 and in particular from 5 to 150.

The fibers that may be used in the composition of the invention may be mineral or organic fibers of synthetic or natural origin. They may be short or long, individual or organized, for example braided, and hollow or solid. They may have any shape, and may especially have a circular or polygonal (square, hexagonal or octagonal) cross section, depending on the intended specific application. In particular, their ends are blunt and/or polished to prevent injury.

In particular, the fibers have a length ranging from 1 μm to 10 mm, preferably from 0.1 mm to 5 mm and better still from 1 mm to 3.5 mm. Their cross section may be within a circle of diameter ranging from 2 nm to 500 μm, preferably ranging from 100 nm to 100 μm and better still from 1 μm to 50 μm. The weight or yarn count of the fibers is often given in denier or decitex, and represents the weight in grams per 9 km of yam. In particular, the fibers may have a yam count chosen in the range from 0.15 to 30 denier and better still from 0.18 to 18 denier.

The fibers can be those used in the manufacture of textiles, and in particular silk fiber, cotton fiber, wool fiber, flax fiber, cellulose fiber extracted in particular from wood, from plants or from algae, rayon fiber, polyamide (Nylon®) fiber, viscose fiber, acetate fiber, in particular rayon acetate fiber, poly(p-phenyleneterephthalamide) (or aramide) fiber, in particular Kevlar® fiber, acrylic polymer fiber, in particular polymethyl methacrylate fiber or poly(2-hydroxyethyl methacrylate) fiber, polyolefin fiber and in particular polyethylene or polypropylene fiber, glass fiber, silica fiber, carbon fiber, in particular in graphite form, polytetrafluoroethylene (such as Teflon®) fiber, insoluble collagen fiber, polyester fiber, polyvinyl chloride fiber or polyvinylidene chloride fiber, polyvinyl alcohol fiber, polyacrylonitrile fiber, chitosan fiber, polyurethane fiber, polyethylene phthalate fiber, and fibers formed from a mixture of polymers such as those mentioned above, for instance polyamidelpolyester fibers.

The fibers used in surgery may also be used, for instance the resorbable synthetic fibers prepared from glycolic acid and caprolactone (Monocryl from Johnson & Johnson); resorbable synthetic fibers of the type which is a copolymer of lactic acid and of glycolic acid (Vicryl from Johnson & Johnson); polyterephthalic ester fibers (Ethibond from Johnson & Johnson) and stainless steel threads (Acier from Johnson & Johnson).

Moreover, the fibers may be treated or untreated at the surface, and coated or uncoated.

In particular, fibers of synthetic origin and in particular organic fibers, such as those used in surgery, are used. Water-insoluble fibers may advantageously be used.

The fibers that may be used in the composition according to the invention may in particular be polyamide fibers, cellulose fibers, poly(p-phenyleneterephthalamide) fibers or polyethylene fibers. Their length (L) may range from 0.1 mm to 5 mm and especially from 0.25 mm to 1.6 mm, and their mean diameter may range from 1 μm to 50 μm. In particular, the polyamide fibers sold by Etablissements P. Bonte under the name “Polyamide 0.9 Dtex 3 mm”, having an average diameter of 6 μm, a yam count of about 0.9 dtex and a length ranging from 0.3 mm to 5 mm, may be used. Cellulose (or rayon) fibers with a mean diameter of 50 μm and a length ranging from 0.5 mm to 6 mm may also be used, for instance those sold under the name “Natural rayon flock fiber RCIBE-N003-M04” by Claremont Flock. Polyethylene fibers, for instance those sold under the name “Shurt Stuff 13 099 F” by Mini Fibers, may also be used.

The composition according to the invention may also comprise “rigid” fibers, as opposed to the fibers mentioned above, which are not rigid fibers.

The rigid fibers, which are initially substantially straight, do not undergo a substantial change in shape when they are placed in a dispersing medium.

The rigid fibers may be chosen from fibers of a synthetic polymer chosen from polyesters, polyurethanes, acrylic polymers, polyolefins, polyamides, in particular non-aromatic polyamides, and aromatic polyimideamides.

Examples of rigid fibers that may be mentioned include:

polyester fibers, such as those obtained by chopping yams sold under the names Fibre 255-100-R11-242T Taille 3 MM (eight-lobed cross section), Fibre 265-34-R11-56T Taille 3 MM (round cross section) and Fibre Coolmax 50-34-591 Taille 3 MM (four-lobed cross section) by Dupont de Nemours;

polyamide fibers, such as those sold under the names Trilobal Nylon 0.120-1.8 DPF; Trilobal Nylon 0.120-18 DPF; Nylon 0.120-6 DPF by Cellusuede Products; or obtained by chopping yams sold under the name Fibre Nomex Brand 430 Taille 3 MM by Dupont de Nemours;

polyimideamide fibers, such as those sold under the names “Kermel” and “Kernel Tech” by RHODIA;

poly(p-phenyleneterephthalamide) (or aramide) sold especially under the name Kevlar® by Dupont de Nemours;

fibers with a multilayer structure comprising alternating layers of polymers chosen from polyesters, acrylic polymers and polyamides, such as those described in EP-A-6,921,217, EP-A-686,858 and U.S. Pat. No. 5,472,798 A. Such fibers are sold under the names “Morphotex” and “Teijin Tetron Morphotex” by Teijin.

Rigid fibers that are particularly advantageous are aromatic polyimideamide fibers.

Polyimideamide yams or fibers that may be used for the compositions according to the invention are described, for example, in the document from R. Pigeon and P. Allard, Chimie Macromoleculaire Appliquée, 40/41 (1974), pages 139-158 (No. 600), or in U.S. Pat. No. 3,802,841 A, FR-A-2,079,785, EP-A1-0,360,728 and EP-A-0,549,494, to which reference may be made.

In particular, the aromatic polyimideamide fibers may be polyimideamide fibers comprising repeating units of formula:

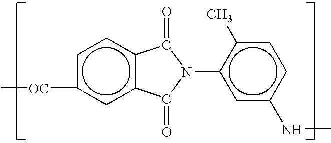

obtained by polycondensation of tolylene diisocyanate and trimellitic anhydride.

The fibers may be present in the composition according to the invention in a content ranging from 0.01% to 10% by weight and especially from 0.05% to 5% by weight relative to the total weight of the composition.

Needless to say, a person skilled in the art will take care to select this (these) ingredient(s) and/or the amount thereof such that the advantageous properties of the composition according to the invention are not, or are not substantially, adversely affected by the envisaged addition.

The compositions according to the invention may also contain one or more common adjuvants such as fragrances, preserving agents, basifying or acidifying agents, texture agents, spreading additives, plasticizers and water-soluble active ingredients commonly used in cosmetic preparations for keratin fibers.

### Preparation Process:

The compositions according to the invention are generally obtained in a conventional manner by hot formation of an emulsion.

More specifically, these compositions are obtained by heating the wax and the surfactant(s) under consideration with an HLB≦8 to a temperature above the melting point of the wax and not greater than 100° C., until the wax has completely melted, followed by gradually adding water and, where appropriate, the gelling polymer and/or surfactants with an HLB>8, brought to a temperature at least equal to the preceding temperature, with stirring until reaching room temperature.

The liposoluble ingredients, for example ceramides, are generally added to the wax before the emulsion is prepared.

Water-soluble ingredients may be added to the water used to make the emulsion, or to the emulsion fmally obtained.

Similarly, secondary ingredients optionally present in the composition are added, depending on the case, either into the starting materials or into the finished composition.

The compositions of the invention may be applied to the eyelashes, using a brush or a comb.

The thickening effect desired in the context of the present invention may moreover be reinforced most particularly by selecting the device for applying the said composition.

In this instance, it is particularly advantageous, in the case of making up the eyelashes, to apply the said composition with a makeup brush as described in FR-2,701,198, FR-2,605,505, EP-792,603 and EP-663,161.

In order to further illustrate the present invention and the advantages thereof, the following specific examples are given, it being understood that same are intended only as illustrative and in nowise limitative. In said examples to follow, all parts and percentages are given by weight, unless otherwise indicated.

The amounts indicated are weight percentages and are expressed relative to the total weight of the composition, unless otherwise indicated.

The rheology measurements were performed on a Haake RheoStress 600 controlled-stress rheometer under the following conditions:

measuring temperature 25° C.,

steady stage of 180 seconds at 25° C. before the start of measurement,

stress sweep from 1 to 10,000 Pa, measuring frequency: 1 Hz.

The gelling polymers and surfactants used are the following:

hydroxyethylcellulose quatemized with 2,3-epoxypropyltrimethylammonium chloride, sold under the name “Ucare Polymer JR 400” by Amerchol (Dow Chemical),

hydroxyethylcellulose sold under the name “Cellobond HEC 5000 A” distributed by Brenntag,

sodium polymethacrylate at 25% in water, unstabilized, sold under the name “Darvan 7” by Vanderbilt,

ethyl acrylate/methyl methacrylate crosslinked copolymer (80/20), as an aqueous 50% dispersion sold under the name “Daitosol 5000 AD” by Daito,

acrylamide/sodium 2-acrylamidomethylpropanesulphonate copolymer as an inverse emulsion at 40% in polysorbate 80/I-C16, sold under the name “Simulgel 600” by SEPPIC,

oxyethylenated glyceryl monostearate (200 EO) sold under the name “Simulsol” by SEPPIC,

oxyethylenated glyceryl monostearate (30 EO) sold under the name “Tagat S” by Degussal Goldschmidt,

AMPS/ethoxylated (25 EO) cetearyl methacrylate copolymer crosslinked with trimethylolpropane triacrylate (TMPTA).

The triethanolamine stearate is prepared in situ by mixing stearic acid and 99% triethanolamine.

The following mascara formulations were prepared:

### Example 1

Formulation A:

| | |
|---|---|
| 2-amino-2-methyl-1,3-propanediol | 0.21% |
| stearic acid (C16-18: 50/50) | 2.49% |
| carnauba wax | 5.77% |
| D-panthenol | 0.50% |
| black iron oxide (CI: 77499) | 3.00% |
| ultramarine blue (CI: 77007) | 4.14% |
| hydroxyethylcellulose quatemized with 2,3-epoxypropyl- | 0.10% |
| trimethylanimonium chloride | |
| hydroxyethylcellulose | 0.88% |
| mixture of polydimethylsiloxane and hydrated silica | 0.15% |
| sodium polymethacrylate at 25% in water, unstabilized | 5.00% |
| gum arabic; polysaccharides; | 3.38% |
| arabinose/galactose/rhamnose/glucuronic acid | |
| propyl p-hydroxybenzoate | 0.20% |
| methyl p-hydroxybenzoate | 0.25% |
| 99% triethanolamine | 1.02% |
| oxyethylenated (20 EO) oxypropylenated (20 PO) | 0.20% |
| polydimethylsiloxane (DP: 170 - viscosity: 1000 cSt) | |
| ethyl acrylate/methyl methacrylate crosslinked copolymer | 4.00% |
| (80/20), as an aqueous 50% dispersion | |
| acrylamide/sodium acrylamido-2-methylpropanesulphonate | 2.00% |
| copolymer as an inverse emulsion | |
| at 40% in polysorbate 80/I-C16 | |
| oxyethylenated (200 EO) glyceryl monostearate | 3.00% |
| trans-isomerized jojoba oil (Simmondsia chinensis) | 15.77% |
| of melting point 45° C. | |
| sterilized demineralized water | qs 100% |

The corresponding formulation has a slippery texture that applies well to the eyelashes. It also has good separating properties on the eyelashes and allows heavy makeup of the eyelashes to be obtained.

### Example 2

Formulation B:

| | |
|---|---|
| 2-amino-2-methyl-1,3-propanediol | 0.21% |
| carnauba wax | 5.77% |
| D-panthenol | 0.50% |
| black iron oxide (CI: 77499) | 3.00% |
| ultramarine blue (CI: 77007) | 4.14% |
| hydroxyethylcellulose quaternized with 2,3-epoxypropyl- | 0.10% |
| trimethylammonium chloride | |
| hydroxyethylcellulose | 0.88% |
| mixture of polydimethylsiloxane and hydrated silica | 0.15% |
| sodium polymethacrylate at 25% in water, unstabilized | 5.00% |
| gum arabic; polysaccharides; | 3.38% |
| arabinose/galactose/rhamnose/glucuronic acid | |
| oxyethylenated (30 EO) glyceryl monosteate | 1.00% |
| propyl p-hydroxybenzoate | 0.20% |
| methyl p-hydroxybenzoate | 0.25% |
| oxyethylenated (20 EO) oxypropylenated (20 PO) | 0.20% |
| polydimethylsiloxane (DP: 170 - viscosity: 1000 cSt) | |
| ethyl acrylate/methyl methacrylate crosslinked copolymer | 4.00% |
| (80/20), as an aqueous 50% dispersion | |
| acrylamide/sodium 2-acrylamidomethylpropanesulphonate | 3.50% |
| copolymer as an inverse emulsion at 40% in | |
| polyborbate 80/I-C16 | |
| oxyethylenated (200 EO) glyceryl monostearate | 3.00% |
| trans-isomerized jojoba oil (Simmondsia chinensis) | 23.28% |
| of melting point 45° C. | |
| sterilized demineralized water | qs 100% |

The formulation thus obtained applies particularly well to the eyelashes. A slippery texture is thus obtained.

### Example 3

Formulation C:

| | |
|---|---|
| 2-amino-2-methyl-1,3-propanediol | 0.21% |
| stearic acid (C16-18: 50/50) | 2.49% |
| carnauba wax | 3.00% |
| D-panthenol | 0.50% |
| 2-phenoxyethanol | 0.50% |
| black iron oxide (CI: 77499) | 3.00% |
| ultramarine blue (CI: 77007) | 4.14% |
| hydroxyethylcellulose quaternized with 2,3-epoxypropyl- | 0.10% |
| trimethylammonium chloride | |
| hydroxyethylcellulose | 0.88% |
| mixture of polydimethylsiloxane and hydrated silica | 0.15% |
| sodium polymethacrylate at 25% in water, unstabilized | 5.00% |
| gum arabic; polysaccharides; | 3.38% |
| arabinose/galactose/rhamnose/glucuronic acid | |
| pure white beeswax | 2.50% |
| glycerol | 3.00% |
| propyl p-hydroxybenzoate | 0.20% |
| methyl p-hydroxybenzoate | 0.25% |
| 99% triethanolamine | 1.02% |
| oxyethylene (20 EO) oxypropylene (20 PO) | 0.20% |
| polydimethylsiloxane (DP: 170 - viscosity: 1000 cSt) | |
| hydrogenated jojoba oil | 6.27% |
| ethyl acrylate/methyl methacrylate crosslinked copolymer | 4.00% |
| (80/20), as an aqueous 50% dispersion | |
| acrylamide/sodium 2-acrylamidomethylpropanesulphonate | 2.00% |
| copolymer as an inverse emulsion at 40% in | |
| polysorbate 80/I-C16 | |
| oxyethylenated (200 EO) glyceryl monostearate | 3.00% |
| trans-isomerized jojoba oil (Simmondsia chinensis) | 6.27% |
| of melting point 45° C. | |
| sterilized demineralized water | qs 100% |

The formulation produces a satisfactory makeup result and good separation of the eyelashes.

### Example 4

Formulation D:

| | |
|---|---|
| 2-amino-2-methyl-1,3-propanediol | 0.21% |
| stearic acid (C16-18: 50/50) | 2.49% |
| camauba wax | 3.00% |
| D-panthenol | 0.50% |
| 2-phenoxyethanol | 0.50% |
| black iron oxide (CI: 77499) | 3.00% |
| ultramarine blue (CI: 77007) | 4.14% |
| hydroxyethylcellulose quaternized with 2,3-epoxypropyl- | 0.10% |
| trimethylammonium chloride | |
| hydroxyethylcellulose | 0.88% |
| mixture of polydimethylsiloxane and hydrated silica | 0.15% |
| sodium polymethacrylate at 25% in water, unstabilized | 5.00% |
| gum arabic; polysaccharides; | 3.38% |
| arabinose/galactose/rhamnose/glucuronic acid | |
| pure white beeswax | 6.50% |
| glycerol | 2.00% |
| propyl p-hydroxybenzoate | 0.20% |
| methyl p-hydroxybenzoate | 0.25% |
| 99% triethanolamine | 1.02% |
| oxyethylene (20 EO) oxypropylene (20 PO) | 0.20% |
| polydimethylsiloxane (DP: 170 - viscosity: 1000 cSt) | |
| hydrogenated jojoba oil | 6.27% |
| ethyl acrylate/methyl methacrylate crosslinked copolymer | 4.00% |
| (80/20), as an aqueous 50% dispersion | |
| acrylamide/sodium 2-acrylamidomethylpropanesulphonate | 2.00% |
| copolymer as an inverse emulsion at 40% in | |
| polysorbate 80/I-C16 | |
| oxyethylenated (200 EO) glyceryl monostearate | 3.00% |
| trans-isomerized jojoba oil (Simmondsia chinensis) | 6.27% |
| of melting point 45° C. | |
| sterilized demineralized water | qs 100% |

The formulation has a creamy texture that is particularly suitable for application by brush, and allows heavy makeup of the eyelashes to be obtained (charging mascara).

### Example 5

Formulation E:

| | |
|---|---|
| 2-amino-2-methyl-1,3-propanediol | 0.21% |
| stearic acid (C16-18: 50/50) | 2.49% |
| camauba wax | 3.00% |
| D-panthenol | 0.50% |
| 2-phenoxyethanol | 0.50% |
| black iron oxide (CI: 77499) | 3.00% |
| ultramarine blue (CI: 77007) | 4.14% |
| hydroxyethylcellulose quaternized with 2,3-epoxypropyl- | 2.30% |
| trimethylammonium chloride | 0.10% |
| hydroxyethylcellulose | 0.88% |
| mixture of polydimethylsiloxane and hydrated silica | 0.15% |
| sodium polymethacrylate at 25% in water, unstabilized | 5.00% |
| gum arabic; polysaccharides; | 3.38% |
| arabinose/galactose/rhamnose/glucuronic acid | |
| pure white beeswax | 5.50% |
| glycerol | 3.00% |
| propyl p-hydroxybenzoate | 0.20% |
| methyl p-hydroxybenzoate | 0.25% |
| 99% triethanolamine | 1.02% |
| oxyethylenated (20 EO) oxypropylenated (20 PO) | 0.20% |
| polydimethylsiloxane (DP: 170 - viscosity: 1000 cSt) | |
| hydrogenated jojoba oil | 6.27% |
| ethyl acrylate/methyl methacrylate crosslinked copolymer | 4.00% |
| (80/20), as an aqueous 50% dispersion | |
| acrylamide/sodium 2-acrylamidomethylpropanesulphonate | 2.00% |
| copolymer as an inverse emulsion at 40% in | |
| polysorbate 80/I-C16 | |
| oxyethylenated (200 EO) glyceryl monostearate | 3.00% |
| trans-isomerized jojoba oil (Simmondsia chinensis) | 6.27% |
| of melting point 45° C. | |
| sterilized demineralized water | qs 100% |

This formulation has good charging and separating properties on the eyelashes. It has a slippery texture that is most particularly suitable for application to the eyelashes.

### Example 6

Formulation F:

| | |
|---|---|
| 2-amino-2-methyl-1,3-propanediol | 0.21% |
| stearic acid (triple-pressed) (C16-18: 50/50) | 2.49% |
| camauba wax | 3.00% |
| D-panthenol | 0.50% |
| 2-phenoxyethanol | 0.50% |
| black iron oxide (CI: 77499) | 5.07% |
| ultramarine blue (CI: 77007) | 2.07% |
| hydroxyethylcellulose quaternized with 2,3-epoxypropyl- | 0.10% |
| trimethylammonium chloride | |
| hydroxyethylcellulose | 0.88% |
| mixture of polydimethylsiloxane and hydrated silica | 0.15% |
| sodium polymethacrylate at 25% in water, unstabilized | 5.00% |
| gum arabic; polysaccharides; | 3.38% |
| arabinose/galactose/rhamnose/glucuronic acid | |
| pure white beeswax | 5.50% |
| glycerol | 3.00% |
| propyl p-hydroxybenzoate | 0.20% |
| methyl p-hydroxybenzoate | 0.25% |
| 99% triethanolamine | 1.02% |
| oxyethylenated (20 EO) oxypropylenated (20 PO) | 0.20% |
| polydimethylsiloxane (DP: 170 - viscosity: 1000 cSt) | |
| hydrogenated jojoba oil | 6.27% |
| cellulose (rayon) fibers | 0.10% |
| ethyl acrylate/methyl methacrylate crosslinked copolymer | 2.00% |
| (80/20), as an aqueous 50% dispersion | |
| acrylamide/sodium 2-acrylamidomethylpropanesulphonate | 2.00% |
| copolymer as an inverse emulsion at 40% in | |
| polysorbate 80/I-C16 | |
| oxyethylenated (200 EO) glyceryl monostearate | 3.00% |
| trans-isomerized jojoba oil (Simmondsia chinensis) | 6.27% |
| of melting point 45° C. | |
| sterilized demineralized water | qs 100% |

The corresponding formulation has a creamy texture and allows heavy makeup of the eyelashes to be obtained.

For the purposes of clarity, the emulsifying systems and the amount of wax used in each of the formulations are specifically identified in Table I below.

The rheological parameters and the solids content were characterized for each of the formulations. They are given in Table H.

TABLE I

| | Nonionic | | Triethanolamine | | | Wax | | |
|---|---|---|---|---|---|---|---|---|
| | Surfactant | | Stearate | | Gelling | (% m) | | |
| | (% m) | | (% m) | | Polymer | | | Isomerized, |
| | PEG | PPEG | Stearic | 99% | (% m) | Carnauba | | Hydrogenated |
| | 30-GS | 200 GS | Acid | Triethanolamine | Simulgel 600 | Wax | Beeswax | Jojoba Waxes |
| A | — | 3.00 | 2.49 | 1.02 | 2.00 | 5.77 | — | 15.77 |
| B | 1.00 | 3.00 | — | — | 3.50 | 5.77 | — | 23.28 |
| C | — | 3.00 | 2.49 | 1.02 | 2.00 | 3.00 | 2.50 | 12.54 |
| D | — | 3.00 | 2.49 | 1.02 | 2.00 | 3.00 | 6.50 | 12.54 |
| E | — | 3.00 | 2.49 | 1.02 | 2.00 | 3.00 | 5.50 | 12.54 |
| F | — | 3.00 | 2.49 | 1.02 | 2.00 | 3.00 | 5.50 | 12.54 |

TABLE II

| | | Modulus | | | Wax |
|---|---|---|---|---|---|
| | Dry Matter | of Rigidity | Threshold | Loss Angle | Particle |
| | (% m) | (10³ Pa) | Stress (Pa) | (°) | Size (μm) |
| A | 47.1 | 10 | 90 | 14 | 0.67 |
| B | 50.5 | 5 | 25 | 20 | 1 |
| C | 46.2 | 9 | 65 | 15 | 0.32 |
| D | 49.2 | 20 | 100 | 16 | 0.82 |
| E | 46.4 | 19 | 100 | 16 | 0.56 |
| F | 46 | 14 | 80 | 18 | 0.6 |

Each patent, patent application, publication and literature article/report cited or indicated herein is hereby expressly incorporated by reference.

While the invention has been described in terms of various specific and preferred embodiments, the skilled artisan will appreciate that various modifications, substitutions, omissions, and changes may be made without departing from the spirit thereof. Accordingly, it is intended that the scope of the present invention be limited solely by the scope of the following claims, including equivalents thereof.

1. A cosmetic composition for making up keratin fibers, comprising:
a waxy phase containing at least one aqueous dispersion of wax particles;
at least one gelling polymer chosen from copolymers of methacrylic acid, and the salts and esters thereof; salts of copolymers of acrylic acid and of acrylamide; the sodium salts of polyhydroxycarboxylic acids; AMPS; AMPS/acrylamide copolymers;
AMPS/polyoxyethylenated alkyl methacrylate copolymers (crosslinked or non-crosslinked); anionic, cationic, amphoteric or nonionic chitin or chitosan polymers; and vinyl polymers;
at least one ionic surfactant having at 25° C. an HLB (hydrophilic-lipophilic balance), in the Griffin sense, of greater than or equal to 8;
wherein the wax particles of the waxy phase have a size, expressed as the mean “effective” diameter by volume D[4.3], of less than or equal to 1 μm;
wherein the cosmetic composition has a plateau Gp modulus of rigidity of greater than or equal to 4,000 Pa and less than 60,000 Pa; and
wherein the composition has a solids content of greater than or equal to 42% by weight relative to the total weight of the composition.

2. The cosmetic composition according to claim 1, having a plateau Gp modulus of rigidity of greater than or equal to 7,000 Pa.

3. The cosmetic composition according to claim 1, wherein the at least one ionic surfactant is chosen from anionic surfactants.

4. The cosmetic composition according to claim 3, wherein the anionic surfactants are chosen from C₁₆-C₃₀ fatty acid salts; polyoxyethylenated fatty acid salts, phosphoric esters and salts thereof; alkyl ether sulphates; sulphosuccinates; isethionates and acylglutamates, and mixtures thereof.

5. The cosmetic composition according to claim 1, wherein the at least one ionic surfactant comprises at least triethanolamine stearate.

6. The cosmetic composition according to claim 1, wherein the at least one ionic surfactant is present in an amount ranging from 0.01% to 30% by weight of the total weight of the composition.

7. The cosmetic composition according to claim 1, wherein the at least one ionic surfactant is present in an amount ranging from 0.1% to 15% by weight of the total weight of the composition.

8. The cosmetic composition according to claim 1, comprising at least one gelling polymer in combination with at least one anionic surfactant with an HLB of greater than or equal to 8 at 25° C., or mixtures thereof.

9. The cosmetic composition according to claim 8, wherein the at least one gelling polymer is chosen from homopolymers or copolymers of acrylic or methacrylic acid or the salts and esters thereof, copolymers of acrylic acid and of acrylamide, polyacrylic acid/alkyl acrylate copolymers, AMPS (polyacrylamidomethylpropanesulphonic acid), AMPS/acrylamide copolymers and AMPS/polyoxyethylenated alkyl methacrylate copolymers, and mixtures thereof.

10. The cosmetic composition according to claim 8, wherein the at least one gelling polymer is present in a solids content ranging from 0.1% to 60% by weight of the total weight of the composition.

11. The composition according to claim 8, wherein the at least one gelling polymer is present in a solids content ranging from 0.5% to 40% by weight of the total weight of the composition.

12. The cosmetic composition according to claim 8, further comprising at least one gelling polymer in combination with at least one nonionic surfactant with an HLB of greater than or equal to 8 at 25° C., or mixtures thereof.

13. The cosmetic composition according to claim 1, further comprising at least one film-forming polymer.

14. The cosmetic composition according to claim 13, wherein the at least one film-forming polymer is chosen from free-radical synthetic polymers, polycondensate synthetic polymers, and polymers of natural origin.

15. The cosmetic composition according to claim 1, having a solids content of greater than or equal to 45.5% by weight.

16. The cosmetic composition according to claim 1, having a solids content of greater than or equal to 48% by weight.

17. The cosmetic composition according to claim 1, wherein the at least one wax is solid and rigid at room temperature and is chosen from animal waxes, plant waxes, mineral waxes, and waxes of synthetic origin.

18. The cosmetic composition according to claim 1, wherein the wax has a melting point of greater than about 45° C.

19. The cosmetic composition according to claim 1, wherein the wax has a melting point of greater than about 55° C.

20. The cosmetic composition according to claim 1, wherein the at least one wax is chosen from hydrocarbon-based waxes; rice wax, carnauba wax, candelilla wax, ouncury wax, esparto grass wax, cork fiber wax, sugar cane wax, Japan wax and sumach wax; montan wax; micro crystalline waxes, paraffins and ozokerites; polyethylene waxes; the waxes obtained by Fisher-Tropsch synthesis; waxy copolymers, and also esters thereof; the waxes obtained by catalytic hydrogenation of animal or plant oils containing linear or branched C₈-C₃₂ fatty chains, for instance hydrogenated jojoba oil, isomerized jojoba oil, hydrogenated sunflower oil, hydrogenated castor oil, hydrogenated coconut oil and hydrogenated lanolin oil, bis(1,1,1-trimethylolpropane) tetrastearate and bis(1,1,1-trimethylolpropane) tetrabehenate.

21. The cosmetic composition according to claim 20, wherein the at least one hydrocarbon-based wax is chosen from beeswax, lanolin wax, and Chinese insect waxes.

22. The cosmetic composition according to claim 1, wherein the waxy phase is present in an amount ranging from 0.1% to 40% by weight.

23. The cosmetic composition according to claim 1, wherein the waxy phase is present in an amount ranging from 5% to 40% by weight.

24. The cosmetic composition according to claim 1, having a flow threshold, measured by oscillatory rheology (ν=1 Hz), ranging from 5 to 3,500 Pa.

25. The cosmetic composition according to claim 1, having a flow threshold, measured by oscillatory rheology (ν=1 Hz), ranging from 20 to 1,000 Pa.

26. The cosmetic composition according to claim 1, further comprising at least one dye stuff and/or at least one filler, wherein
the at least one dye stuff is present in the composition in an amount ranging from 0.01% to 30% by weight relative to the total weight of the composition; and
the at least one filler is present in the composition in an amount ranging from 0.1% to 25% by weight relative to the total weight of the composition.

27. The cosmetic composition according to claim 1, having a plateau elasticity δₚ ranging from 1° to 45°.

28. The cosmetic composition according to claim 1, having a plateau elasticity δₚ ranging from 5° to 30°.

29. The cosmetic composition according to claim 1, further comprising at least one nonionic surfactant with an HLB value of greater than or equal to 8 at 25° C.

30. The cosmetic composition according to claim 29, wherein the at least one nonionic surfactant with an HLB of greater than or equal to 8 is chosen from oxyethylenated and/or oxypropylenated fatty alcohol ethers, fatty acid esters of polyethylene glycol, oxyethylenated and/or oxypropylenated glycerol ethers, fatty acid esters of oxyethylenated and/or oxypropylenated sorbitol ethers, and copolymers of propylene oxide and of ethylene oxide, and mixtures thereof.

31. The cosmetic composition according to claim 30, wherein the at least one nonionic surfactant is chosen from oxyethylenated ethers of cetearyl alcohol containing 30 oxyethylene groups, oxyethylenated ethers of a mixture of C₁₂-C₁₅ fatty alcohols comprising 7 oxyethylene groups, PEG-SO stearate, PEG-40 stearate, PEG-200 glyceryl stearate, polysorbate 60, glyceryl stearate polyethoxylated with 30 ethylene oxide groups, glyceryl oleate polyethoxylated with 30 ethylene oxide groups, glyceryl cocoate polyethoxylated with 30 ethylene oxide groups, glyceryl isostearate polyethoxylated with 30 ethylene oxide groups, glyceryl laurate polyethoxylated with 30 ethylene oxide groups, dimethicone copolyol, and dimethicone copolyol benzoate.

32. The cosmetic composition according to claim 29, wherein the at least one nonionic surfactant with an HLB of greater than or equal to 8 is present in an amount ranging from 0.01% to 40% by weight of the total weight of the composition.

33. The cosmetic composition according to claim 29, wherein the at least one nonionic surfactant with an HLB of greater than or equal to 8 is present in an amount ranging from 0.1% to 25% by weight of the total weight of the composition.

34. The cosmetic composition according to claim 29, wherein the at least one ionic surfactant and the at least one nonionic surfactant are present in the composition in an amount ranging from 0.1% to 40% by weight relative to the total weight of the composition.

35. The cosmetic composition according to claim 34, wherein the at least one ionic surfactant and the at least one nonionic surfactant are present in the composition in an amount ranging from 0.5% to 20% by weight relative to the total weight of the composition.

36. A cosmetic composition for making up keratin fibers, comprising:
a waxy phase containing at least one aqueous dispersion of wax particles;
at least one gelling polymer chosen from copolymers of methacrylic acid, and the salts and esters thereof; and AMPS/acrylamide copolymers;
at least one ionic surfactant having at 25° C. an HLB (hydrophilic-lipophilic balance), in the Griffin sense, of greater than or equal to 8;
wherein the wax particles of the waxy phase have a size, expressed as the mean “effective” diameter by volume D[4.3], of less than or equal to 1 μm;
wherein the cosmetic composition has a plateau Gp modulus of rigidity of greater than or equal to 4,000 Pa and less than 60,000 Pa; and
wherein the composition has a solids content of greater than or equal to 42% by weight relative to the total weight of the composition.

37. A regime or regimen for the charging makeup of keratin fibers, comprising topically applying thereon an effective amount of the cosmetic composition comprising:
a waxy phase containing at least one aqueous dispersion of wax particles;
at least one gelling polymer chosen from copolymers of methacrylic acid, and the salts and esters thereof; and AMPS/acrylamide copolymers;
at least one ionic surfactant having at 25° C. an HLB (hydrophilic-lipophilic balance), in the Griffin sense, of greater than or equal to 8;
wherein the wax particles of the waxy phase have a size, expressed as the mean “effective” diameter by volume D[4.3], of less than or equal to 1 mm;
wherein the cosmetic composition has a plateau Gp modulus of rigidity of greater than or equal to 4,000 Pa and less than 60,000 Pa; and
wherein the composition has a solids content of greater than or equal to 42% by weight relative to the total weight of the composition.

38. A regime or regimen for the charging makeup of keratin fibers, comprising topically applying thereon an effective amount of the cosmetic composition comprising:
a waxy phase containing at least one aqueous dispersion of wax particles;
at least one gelling polymer chosen from copolymers of methacrylic acid, and the salts and esters thereof; salts of copolymers of acrylic acid and of acrylamide; the sodium salts of polyhydroxycarboxylic acids; AMPS; AMPS/acrylamide copolymers;
AMPS/polyoxyethylenated alkyl methacrylate copolymers (crosslinked or non-crosslinked); anionic, cationic, amphoteric or nonionic chitin or chitosan polymers; and vinyl polymers;
at least one ionic surfactant having at 25° C. an HLB (hydrophilic-lipophilic balance), in the Griffin sense, of greater than or equal to 8;
wherein the wax particles of the waxy phase have a size, expressed as the mean “effective” diameter by volume D[4.3], of less than or equal to 1 μm;
wherein the cosmetic composition has a plateau Gp modulus of rigidity of greater than or equal to 4,000 Pa and less than 60,000 Pa; and
wherein the composition has a solids content of greater than or equal to 42% by weight relative to the total weight of the composition.

39. The regime or regimen of claim 38, comprising the charging makeup of the eyelashes and/or eyebrows.

40. The regime or regimen for the charging makeup of keratin fibers of claim 38, further comprising at least one nonionic surfactant with an HLB value of greater than or equal to 8 at 25° C.

41. A process for preparing a cosmetic composition for making up keratin fibers comprising
bringing a starting composition comprising wax to a first temperature above the melting point of the wax and not greater than 100° C., thereby melting the wax to form a melted wax composition;
gradually adding water, at least one gelling polymer, and at least one ionic surfactant to the melted wax composition to form a mixture;
bringing the mixture to a second temperature greater than or equal to the first temperature; and
stirring the mixture while cooling the mixture to room temperature, thereby obtaining the cosmetic composition,
wherein the cosmetic composition comprises a waxy phase containing at least one aqueous dispersion of wax particles;
wherein the wax particles have a size, expressed as the mean “effective” diameter by volume D[4.3], of less than or equal to 1 μm;
wherein said at least one gelling polymer is chosen from copolymers of methacrylic acid, and the salts and esters thereof; salts of copolymers of acrylic acid and of acrylamide; the sodium salts of polyhydroxycarboxylic acids; AMPS; AMPS/acrylamide copolymers; AMPS/polyoxyethylenated alkyl methacrylate copolymers (crosslinked or non-crosslinked); anionic, cationic, amphoteric or nonionic chitin or chitosan polymers; and vinyl polymers;
wherein said at least one ionic surfactant has, at 25° C., an HLB (hydrophilic-lipophilic balance), in the Griffin sense, of greater than or equal to 8;
wherein the cosmetic composition has a solids content of greater than or equal to 42% by weight relative to the total weight of the cosmetic composition; and
wherein the cosmetic composition has a plateau Gp modulus of rigidity of greater than or equal to 4,000 Pa and less than 60,000 Pa.

42. A cosmetic composition for making up keratin fibers prepared by the process of claim 41.

43. The cosmetic composition according to claim 42, wherein the at least one ionic surfactant is chosen from C₁₆-C₃₀ fatty acid salts; polyoxyethylenated fatty acid salts, phosphoric esters and salts thereof; alkyl ether sulphates; sulphosuccinates; isethionates and acylglutamates; and mixtures thereof.

44. The cosmetic composition according to claim 42, wherein the wax has a melting point of greater than about 45° C.

45. The cosmetic composition according to claim 42, further comprising at least one nonionic surfactant with an HLB value of greater than or equal to 8 at 25° C.

46. The cosmetic composition according to claim 45, wherein the at least one nonionic surfactant with an HLB of greater than or equal to 8 is chosen from oxyethylenated and/or oxypropylenated fatty alcohol ethers, fatty acid esters of polyethylene glycol, oxyethylenated and/or oxypropylenated glycerol ethers, fatty acid esters of oxyethylenated and/or oxypropylenated sorbitol ethers, and copolymers of propylene oxide and of ethylene oxide, and mixtures thereof.

47. The cosmetic composition according to claim 42, wherein the at least one gelling polymer is chosen from copolymers of methacrylic acid, and the salts and esters thereof; and AMPS/acrylamide copolymers.
